(19) United States
(12) Patent Application Publication
(10) Pub. No.: US 2024/0076607 A1
(43) Pub. Date: Mar. 7, 2024

(54) PROCESS AND PRODUCT THEREOF

(71) Applicant: 3F BIO LTD, Glasgow (GB)

(72) Inventors: Robert James Taylor LAIRD, Glasgow (GB); Yogeshwar Buadass CHANDELIA, Glasgow (GB); Paul Andrew HUDMAN, Glasgow (GB)

(21) Appl. No.: 18/262,207

(22) PCT Filed: Jan. 21, 2022
(86) PCT No.: PCT/GB2022/050174

§ 371 Date: Jul. 20, 2023

(30) Foreign Application Priority Data
Jan. 21, 2021 (GB) 2100819.8

(51) Int. Cl. C12N 1/14; A23J 1/00; A23J 3/20; C07K 14/37; C12N 1/06

(52) U.S. Cl. CPC C12N 1/14; A23J 1/008; A23J 3/20; C07K 14/37; C12N 1/066

(57) ABSTRACT

There is described a process for producing at least one of mycoprotein and the components thereof, the process comprising: (i) providing a fermentation media suitable for producing mycoprotein; (ii) fermenting the fermentation media to obtain a mixture comprising mycoprotein; (iii) separating the mycoprotein from the mixture to obtain a mycoprotein phase; and (iv) mechanically disrupting the cell walls of the mycoprotein in the mycoprotein phase thereby releasing at least some of the mycoprotein cell contents. There is also described at least one of mycoprotein and the components thereof obtainable, obtained or directly obtained from the process. Further described is a composition comprising at least one of mycoprotein and the components thereof, wherein the composition is non-fibrous. The composition may comprise at least one of: protein obtained from mycoprotein, and amino acids derived from protein obtained from mycoprotein.

### FIELD OF INVENTION

The present invention relates to a process for producing mycoprotein and the product thereof. In particular, the present invention relates to an efficient, low energy and/or cost-effective process for producing mycoprotein and the product thereof.

### BACKGROUND

Mycoprotein is a form of single-cell protein that is typically used as a food product or ingredient. It is conventionally produced by aerobic fermentation of a carbohydrate source using filamentous fungi, such as Fusarium venenatum.

GB2137226A describes a process for producing mycoprotein by continuous aerobic fermentation using Fusarium graminearum in a culture medium containing all necessary growth promoting nutrient substances. After the mycoprotein is grown by aerobic fermentation, a heat treatment step is required to reduce the content of nucleic acid, such as RNA, present in the mycoprotein product.

GB1440642A describes a method used to reduce the content of RNA in a mycoprotein product. A heat treatment step is performed on material that has been harvested by filtration, washed, and then resuspended in water.

The production of mycoprotein is expensive. The high cost is associated with the use of refined feedstock (typically glucose syrup), high water usage, high energy costs associated with aerobic fermentation, and high plant operating costs. Also, whilst mycoprotein is a popular meat substitute, due to its fibrous structure it has to date had limited applications in other foodstuffs.

WO 2016/063053 describes a process for the coproduction of mycoprotein and ethanol. In particular, mycoprotein is produced by aerobic fermentation of Fusarium species. The fermentation broth undergoes a heat treatment step to reduce RNA content and is then separated to provide mycoprotein paste and spent mycoprotein fermentation liquor. The spent mycoprotein fermentation liquor is then fed into an anaerobic fermentation process to provide ethanol.

However, the heat treatment step used to reduce the RNA content in typical mycoprotein production processes can result in a loss of up to 30% of the mycoprotein that has just been produced. This increases the cost of producing mycoprotein, which is further exacerbated in that the heat treatment step used has high energy costs and high processing times. The resulting mycoprotein has a filamentous or fibrous hyphal nature (approximate length 300-700 μm) providing a meat-like texture to products formulated with the mycoprotein.

Therefore, it is an object of the present invention to provide an efficient, low energy and cost-effective process for obtaining mycoprotein. It is a further object of the invention to mitigate the loss of mycoprotein when reducing its RNA content. It is a further object of the invention to provide a mycoprotein foodstuff suitable for use in foodstuffs other than meat substitutes.

It is a further object of the invention to mitigate at least some of the disadvantages of the prior art. Further objects of the invention will be apparent from reading the following.

### DISCLOSURE OF INVENTION

According to a first aspect of the invention, there is provided a process for producing at least one of mycoprotein and the components thereof, the process comprising:
(i) providing a fermentation media suitable for producing mycoprotein;
(ii) fermenting the fermentation media to obtain a mixture comprising mycoprotein;
(iii) separating the mycoprotein from the mixture to obtain a mycoprotein phase; and
(iv) mechanically disrupting the cell walls of the mycoprotein in the mycoprotein phase thereby releasing at least some of the mycoprotein cell contents.

The mixture may comprise mycoprotein and at least partially spent fermentation media.

Typically, fermenting the fermentation media is to obtain a mycoprotein biomass, the mycoprotein biomass comprising mycoprotein.

The process may comprise:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase; and
(v) mechanically disrupting the cell walls of the mycoprotein in the mycoprotein phase thereby releasing at least some of the mycoprotein cell contents.

By the term “mycoprotein” is meant a fungal biomass comprised of mycoprotein cells, the mycoprotein cells comprising protein. The term mycoprotein may refer to the mycoprotein cells prior to and/or after RNA reduction.

By the term “mycoprotein and the components thereof” is meant mycoprotein and/or one or more of the component parts of mycoprotein including, but not limited to: mycoprotein cellular materials and the constituent parts of those cellular materials. The constituent parts of the cellular materials may include, for example, amino acids from the proteolysis of protein from the mycoprotein. It will be understood that the components of the mycoprotein have been obtained therefrom.

By the term “mycoprotein cellular materials” is meant the components that form mycoprotein cells including, but not limited to: protein, RNA, chitin, glucans, chitosans, nucleic acids, sterols, amino acids, lipids, glycerides, vitamins, enzymes, organic acids, peptides, protein fragments, storage carbohydrates and minerals. Minerals include, but are not limited to, phosphorus, magnesium, manganese and zinc. Enzymes include, but are not limited to, ribonuclease, phosphodiesterase, protease, trehalase and tyrosinase. Peptides include, but are not limited to, dipeptides, tripeptides and oligopeptides. Storage carbohydrates include, but are not limited to, trehalose and glycogen.

By the term “media” is meant a solid, liquid or semi-solid designed to support the growth of microorganisms.

By the term “fermentation media” is meant media suitable for fermentation. For example, media comprising the components required to support the growth of microorganisms used for fermentation.

The fermentation media may be an aqueous fermentable broth suitable for producing mycoprotein.

The fermentation media may comprise water, a carbohydrate, a source of nitrogen and nutrients. The nutrients may be suitable for producing mycoprotein. The nutrients may be selected from one or more of the group consisting of: salts, vitamins and trace metals.

The salts may be selected from one or more of the group consisting of: potassium sulphate, potassium phosphate, magnesium sulphate, manganese chloride, calcium acetate, calcium chloride, iron sulphate, iron chloride, zinc sulphate, zinc chloride, copper sulphate, copper chloride, cobalt chloride, ammonium chloride, sodium molybdate, ammonium hydroxide, ammonium phosphate and choline salts.

By the term “partially spent fermentation media” is meant media that has undergone fermentation. The partially spent media may comprise at least a portion of the carbohydrate and/or nutrients from the original fermentation media.

The process may be a continuous process.

The step of separating the mixture comprising mycoprotein and at least partially spent fermentation media into a mycoprotein phase and an at least partially spent fermentation media phase may comprise one or more of centrifugation and filtration.

The centrifugation may be decanter and/or disc stack centrifugation. However, any suitable centrifugation means and/or apparatus may be used.

The filtration may be cross flow filtration. However, any suitable filtration means and/or apparatus may be used.

The at least partially spent media phase may be centrate, filtrate, or the like.

The carbohydrate in the fermentation media prior to fermentation may be in excess.

The nutrients in the fermentation media prior to fermentation may be in excess and/or at a pre-determined concentration.

The specific growth rate of the mycoprotein may be between approximately 0.17 h⁻¹ and approximately 0.2 h⁻¹.

The specific growth rate of the mycoprotein may be between approximately 0.16 h⁻¹ and approximately 0.2 h⁻¹.

Mechanically disrupting the cell walls of the mycoprotein may comprise applying mechanical force to the mycoprotein cell walls, optionally wherein the mechanical force is a shearing force.

Mechanically disrupting the cell walls may comprise using one or more of: high pressure homogenisation, microfluidisation, a French pressure cell press, sonication, ultrasonication, bead mills, a Hughes press and an X-press, optionally wherein mechanically disrupting the cell walls comprises using one or more of: high pressure homogenisation and microfluidisation; optionally high pressure homogenisation.

The mycoprotein phase may be an aqueous suspension of mycoprotein, optionally wherein the mycoprotein phase comprises from approximately 1.5% w/w to approximately 30% w/w mycoprotein, optionally between approximately 10% w/w and approximately 20% w/w mycoprotein.

The process may comprise the further step of reducing the viscosity of the mycoprotein phase, optionally wherein reducing viscosity comprises one or more of: high shear mixing and blending. Reducing the viscosity may comprise the application of thinning agents to the mycoprotein phase. Reducing the viscosity may comprise using one or more of: high shear mixing, blending and thinning agents. Thinning agents include, but are not limited to, glycerol and glycol.

The mechanical disruption of the cell walls may be carried out after the reduction in viscosity of the mycoprotein phase.

The step of reducing the viscosity of the mycoprotein phase may be carried out before mechanically disrupting the cell walls of the mycoprotein.

The step of reducing the viscosity of the mycoprotein phase may comprise reducing the hyphal length of the mycoprotein in the mycoprotein phase, optionally wherein reducing hyphal length comprises one or more of: high shear mixing and blending, optionally wherein the hyphal length is reduced to less than approximately 50 μm.

Mechanically disrupting the cell walls of the mycoprotein in the mycoprotein phase may be carried out without the application of heat. Mechanically disrupting the cell walls of the mycoprotein in the mycoprotein phase may be carried out without the use and/or application of any external heat source.

The process may be carried out without applying one or more of chemical and enzymatic cell disruption.

The mechanical disruption may exclude one or more of chemical and enzymatic disruption.

On releasing the cell contents of the mycoprotein in the mycoprotein phase, there may be formed a mixture comprising mycoprotein cellular materials, the mycoprotein cellular materials comprising one or more of: protein and RNA.

The mycoprotein cellular materials may comprise other cellular materials, optionally wherein the other cellular materials comprise one or more of: chitin, glucans, chitosans, nucleic acids, sterols, amino acids, lipids, glycerides, vitamins and minerals. Optionally, wherein the other cellular materials comprise one or more of: chitin, glucans, chitosans, nucleic acids, sterols, amino acids, lipids, glycerides, vitamins, enzymes, organic acids, peptides, protein fragments, storage carbohydrates and minerals.

The process may comprise the further step of denaturing the enzymes in the mixture comprising mycoprotein cellular materials. Denaturing the enzymes may comprise applying mechanical and/or physicochemical stress to the mixture comprising mycoprotein cellular materials. The enzyme denaturation step may comprise one or more of: pasteurisation, sterilisation, secondary membrane processing, agitation, radiation, freezing, application of high pressure, application of acid, application of base, application of inorganic salt, application of organic solvent, and application of a fermenting agent that reduces fermentable sugars. The enzyme denaturation may comprise pasteurisation of the mixture comprising mycoprotein cellular materials. The enzyme denaturation may comprise the application of heat to the mixture comprising mycoprotein cellular materials, optionally wherein the application of heat comprises one or more of pasteurisation, sterilisation, and ultra-high temperature processing (UHT). The enzyme denaturation may comprise pasteurisation, optionally wherein the pasteurisation comprises the application of heat to the mixture comprising mycoprotein cellular materials.

The step of denaturing the enzymes may be carried out after mechanically disrupting the cell walls of the mycoprotein.

The process may comprise the further step of separating the protein from the mixture comprising mycoprotein cellular materials.

The step of separating the protein from the mixture comprising mycoprotein cellular materials may be carried out after the enzyme denaturation step. The step of separating the protein from the mixture comprising mycoprotein cellular materials may be carried out at the same time as the enzyme denaturation step. The step of separating the protein from the mixture comprising mycoprotein cellular materials may be carried out in parallel with the enzyme denaturation step.

One or more of the other cellular materials may be separated from the mixture comprising mycoprotein cellular materials. For example, chitin may be separated from the mixture comprising mycoprotein cellular materials.

The process may comprise the further step of proteolysis of the protein into constituent amino acids.

The process may comprise the further step of separating the protein from the other cellular materials.

Releasing the RNA from the mycoprotein cells may cause denaturing of the RNA, optionally wherein the amount of RNA is reduced to less than approximately 2% w/w on a dry weight basis, optionally wherein the amount of RNA is reduced to less than approximately 1% w/w on a dry weight basis.

Releasing the RNA from the mycoprotein cells may cause denaturing of the RNA, optionally wherein the amount of viable RNA is reduced to less than approximately 2% w/w on a dry weight basis, optionally wherein the amount of viable RNA is reduced to less than approximately 1% w/w on a dry weight basis.

By the term “viable RNA” is meant RNA that has not been denatured.

When the RNA is released from the mycoprotein cell it spontaneously denatures. This is because outside of the mycoprotein cell, the RNA is inherently unstable. Denaturing of the RNA can also be caused by the action of ribonuclease enzymes that have also been released from the mycoprotein cells or other factors such as the interaction of the RNA with UV light or with other components of the mixture comprising mycoprotein cellular materials.

The denaturing of the RNA may occur without the application of heat.

The mixture comprising mycoprotein cellular materials may comprise less than approximately 2% RNA w/w on a dry weight basis, optionally less than approximately 1% RNA w/w on a dry weight basis. The total cellular RNA originally present in mycoprotein is typically approximately 10% w/w on a dry weight basis.

The mixture comprising mycoprotein cellular materials may comprise less than approximately 2% viable RNA w/w on a dry weight basis, optionally less than approximately 1% viable RNA w/w on a dry weight basis.

The denaturing of the RNA may occur at ambient temperature, where ambient temperature is up to approximately 40° C.

The denaturing of the RNA occurs at ambient temperature without the use and/or application of any external heat source. The mixture comprising mycoprotein cellular materials may be held in a fermentation tank or a buffer tank for a period of time to ensure that RNA content has reduced to a suitable level. The period of time may be up to approximately 60 minutes optionally up to approximately 15 minutes.

The fermentation media may comprise a carbohydrate suitable for producing mycoprotein, optionally wherein the carbohydrate is a sugar, optionally wherein the carbohydrate is glucose, sucrose or a source thereof.

The fermentation media may be obtained from a feedstock. The feedstock may be at least one of a starch-based feedstock and a sugar-based feedstock. The starch-based feedstock may be selected from one or more of the group consisting of a grain, cassava and potatoes. The feedstock may be a grain. The grain may be at least one of wheat, maize, buckwheat, rye, barley, millet and rice. The sugar-based feedstock may be selected from one or more of the group consisting of sugarcane, sugar beets and sweet sorghum. The feedstock may be sugarcane. The feedstock may be subjected to one or more of milling, grinding, gelatinisation, liquefaction and saccharification before the step of introducing the fermentation media to the fermentation vessel. The fermentation media may be an aqueous fermentable broth comprising hydrolysed starch.

The fermentation vessel may be an aerobic fermentation vessel.

The fermentation media may be fermented with a microorganism to obtain a mixture comprising mycoprotein and at least partially spent fermentation media, optionally wherein the microorganism is filamentous fungi, optionally wherein the filamentous fungi is selected from one or more of the group consisting of Aspergillus species, Rhizopus species and Fusarium species, optionally wherein the microorganism is Fusarium venenatum.

The process may comprise the further step of washing the mycoprotein phase with water so-forming a mixture comprising mycoprotein and water, wherein the wash step is carried out after the separation step and before the mechanical cell disruption step, optionally wherein the wash step comprises adding the mycoprotein phase to a wash tank and adding water.

The process may comprise the further step of removing water from the so-formed mixture comprising mycoprotein and water.

The washing, water removal and dilution steps, may provide a mycoprotein phase comprising between approximately 1.5% w/w and approximately 30% w/w mycoprotein, optionally between approximately 10% w/w and approximately 20% w/w.

According to a second aspect of the invention, there is provided at least one of mycoprotein and the components thereof obtainable, obtained or directly obtained by the process of the first aspect.

According to a third aspect of the invention, there is provided a composition comprising at least one of mycoprotein and the components thereof, wherein the composition is non-fibrous, optionally wherein the composition comprises at least one of: protein obtained from mycoprotein, and amino acids derived from protein obtained from mycoprotein. The composition may comprise at least one of mycoprotein and the components thereof, wherein the composition is non-fibrous, optionally wherein the composition comprises at least one of: protein obtained from mycoprotein, peptides obtained from mycoprotein, oligopeptides obtained from mycoprotein, and amino acids derived from protein obtained from mycoprotein.

According to a fourth aspect of the invention, there is provided a process for producing at least one of mycoprotein and the components thereof, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase; and
(v) mechanically disrupting the cell walls of the mycoprotein in the mycoprotein phase thereby releasing at least some of the mycoprotein cell contents.

According to a fifth aspect of the invention, there is provided a process for reducing the RNA content of mycoprotein, the process comprising:
(i) providing mycoprotein; and
(ii) mechanically disrupting the mycoprotein cell walls thereby releasing at least some of the mycoprotein cell contents.

Optionally, on releasing the cell contents of the mycoprotein in the mycoprotein phase, there is formed a mixture comprising mycoprotein cellular materials, the mycoprotein cellular materials comprising one or more of: protein and RNA.

Releasing the RNA from the mycoprotein cells causes denaturing of the RNA.

The denaturing of the RNA may occur without the application of heat.

According to a sixth aspect of the invention, there is provided a process for preparing a non-fibrous mycoprotein, the process comprising:
(i) providing mycoprotein; and
(ii) mechanically disrupting the mycoprotein cell walls thereby releasing at least some of the mycoprotein cell contents.

According to a seventh aspect of the invention, there is provided a process for releasing at least some of the contents of a mycoprotein cell, the process comprising:
(i) providing mycoprotein; and
(ii) mechanically disrupting the mycoprotein cell walls thereby releasing at least some of the mycoprotein cell contents.

According to an eight aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase;
(v) reducing the viscosity of the mycoprotein phase; and
(vi) mechanically disrupting the cell walls of the mycoprotein in the mycoprotein phase thereby releasing at least some of the mycoprotein cell contents.

Optionally the mechanical disruption of the cell walls is carried out after the reduction in viscosity of the mycoprotein phase.

The step of reducing the viscosity of the mycoprotein phase may be carried out before mechanically disrupting the cell walls of the mycoprotein.

According to a ninth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase;
(v) reducing the hyphal length of the mycoprotein in the mycoprotein phase; and
(vi) mechanically disrupting the cell walls of the mycoprotein in the mycoprotein phase thereby releasing at least some of the mycoprotein cell contents.

Optionally the mechanical disruption of the cell walls is carried out after the reduction in hyphal length of the mycoprotein.

The step of reducing the hyphal length of the mycoprotein may be carried out before mechanically disrupting the cell walls of the mycoprotein.

According to a tenth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase;
(v) reducing the hyphal length of the mycoprotein in the mycoprotein phase thereby reducing the viscosity of the mycoprotein in the mycoprotein phase; and
(vi) mechanically disrupting the cell walls of the mycoprotein in the mycoprotein phase thereby releasing at least some of the mycoprotein cell contents.

According to an eleventh aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase; and
(v) mechanically disrupting the cell walls of the mycoprotein in the mycoprotein phase thereby releasing at least some of the mycoprotein cell contents.

wherein on releasing the cell contents of the mycoprotein in the mycoprotein phase there is formed a mixture comprising mycoprotein cellular materials, the mycoprotein cellular materials comprising one or more of: protein and RNA, and wherein releasing the RNA from the mycoprotein cells causes denaturing of the RNA.

The denaturing of the RNA occurs without the application of heat.

According to a twelfth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase; and
(v) mechanically disrupting the cell walls of the mycoprotein in the mycoprotein phase thereby releasing at least some of the mycoprotein cell contents.

wherein on releasing the cell contents of the mycoprotein in the mycoprotein phase there is formed a mixture comprising mycoprotein cellular materials, the mycoprotein cellular materials comprising one or more of: protein and RNA, and the process comprises the further step of separating the protein from the mixture comprising mycoprotein cellular materials.

The process may comprise the further step of breaking down the protein into its constituent amino acids.

The process may comprise the further step of separating the protein from the other cellular materials.

According to a thirteenth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase;
(v) reducing the viscosity of the mycoprotein phase; and
(vi) mechanically disrupting the cell walls of the mycoprotein in the mycoprotein phase thereby releasing at least some of the mycoprotein cell contents;

wherein on releasing the cell contents of the mycoprotein in the mycoprotein phase there is formed a mixture comprising mycoprotein cellular materials, the mycoprotein cellular materials comprising one or more of: protein and RNA, and wherein releasing the RNA from the mycoprotein cells causes denaturing of the RNA.

The denaturing of the RNA occurs without the application of heat.

According to a fourteenth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase;
(v) reducing the viscosity of the mycoprotein phase; and
(vi) mechanically disrupting the cell walls of the mycoprotein in the mycoprotein phase thereby releasing at least some of the mycoprotein cell contents;

wherein on releasing the cell contents of the mycoprotein in the mycoprotein phase there is formed a mixture comprising mycoprotein cellular materials, the mycoprotein cellular materials comprising one or more of: protein and RNA, and the process comprises the further step of separating the protein from the mixture comprising mycoprotein cellular materials.

The process may comprise the further step of breaking down the protein into its constituent amino acids.

The process may comprise the further step of separating the protein from the other cellular materials.

According to a fifteenth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase; and
(v) mechanically disrupting the cell walls of the mycoprotein in the mycoprotein phase thereby releasing at least some of the mycoprotein cell contents;

wherein on releasing the cell contents of the mycoprotein in the mycoprotein phase there is formed a mixture comprising mycoprotein cellular materials, the mycoprotein cellular materials comprising one or more of: protein, RNA and other cellular materials.

The other cellular materials may comprise one or more of: chitin, glucans, chitosans, nucleic acids, sterols, amino acids, lipids, glycerides, vitamins, enzymes, organic acids, peptides, protein fragments, storage carbohydrates and minerals.

The process may comprise the further step of denaturing the enzymes in the mixture comprising mycoprotein cellular materials. The enzyme denaturation step may comprise one or more of: pasteurisation, sterilisation, secondary membrane processing, agitation, radiation, freezing, application of high pressure, application of acid, application of base, application of inorganic salt, application of organic solvent, and application of a fermenting agent that reduces fermentable sugars. The enzyme denaturation may comprise pasteurisation of the mixture comprising mycoprotein cellular materials. The enzyme denaturation may comprise the application of heat to the mixture comprising mycoprotein cellular materials, optionally wherein the application of heat comprises one or more of pasteurisation, sterilisation, and ultra-high temperature processing (UHT). The enzyme denaturation may comprise pasteurisation, optionally wherein the pasteurisation comprises the application of heat to the mixture comprising mycoprotein cellular materials.

The step of denaturing the enzymes may be carried out after mechanically disrupting the cell walls of the mycoprotein.

The process may comprise the further step of separating the protein from the mixture comprising mycoprotein cellular materials. The step of separating the protein from the mixture comprising mycoprotein cellular materials may be carried out after the enzyme denaturation step. The step of separating the protein from the mixture comprising mycoprotein cellular materials may be carried out in parallel with the enzyme denaturation step.

According to a sixteenth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase;
(v) reducing the viscosity of the mycoprotein phase; and
(vi) mechanically disrupting the cell walls of the mycoprotein in the mycoprotein phase thereby releasing at least some of the mycoprotein cell contents;

wherein on releasing the cell contents of the mycoprotein in the mycoprotein phase there is formed a mixture comprising mycoprotein cellular materials, the mycoprotein cellular materials comprising one or more of: protein, RNA and other cellular materials.

The process may comprise the further step of denaturing the enzymes in the mixture comprising mycoprotein cellular materials. The enzyme denaturation step may comprise one or more of: pasteurisation, sterilisation, secondary membrane processing, agitation, radiation, freezing, application of high pressure, application of acid, application of base, application of inorganic salt, application of organic solvent, and application of a fermenting agent that reduces fermentable sugars. The enzyme denaturation may comprise pasteurisation of the mixture comprising mycoprotein cellular materials. The enzyme denaturation may comprise the application of heat to the mixture comprising mycoprotein cellular materials, optionally wherein the application of heat comprises one or more of pasteurisation, sterilisation, and ultra-high temperature processing (UHT). The enzyme denaturation may comprise pasteurisation, optionally wherein the pasteurisation comprises the application of heat to the mixture comprising mycoprotein cellular materials.

The step of denaturing the enzymes may be carried out after mechanically disrupting the cell walls of the mycoprotein.

The process may comprise the further step of separating the protein from the mixture comprising mycoprotein cellular materials. The step of separating the protein from the mixture comprising mycoprotein cellular materials may be carried out after the enzyme denaturation step. The step of separating the protein from the mixture comprising mycoprotein cellular materials may be carried out in parallel with the enzyme denaturation step.

According to a seventeenth aspect of the invention, there is provided a process for producing mycoprotein, the process comprising:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase;
(v) reducing the viscosity of the mycoprotein phase; and
(vi) mechanically disrupting the cell walls of the mycoprotein in the mycoprotein phase thereby releasing at least some of the mycoprotein cell contents;

wherein on releasing the cell contents of the mycoprotein in the mycoprotein phase there is formed a mixture comprising mycoprotein cellular materials, the mycoprotein cellular materials comprising one or more of: protein, RNA and enzymes, and wherein the process comprises the further step of denaturing the enzymes in the mixture comprising mycoprotein cellular materials.

The enzyme denaturation step may comprise one or more of: pasteurisation, sterilisation, secondary membrane processing, agitation, radiation, freezing, application of high pressure, application of acid, application of base, application of inorganic salt, application of organic solvent, and application of a fermenting agent that reduces fermentable sugars. The enzyme denaturation may comprise pasteurisation of the mixture comprising mycoprotein cellular materials. The enzyme denaturation may comprise the application of heat to the mixture comprising mycoprotein cellular materials, optionally wherein the application of heat comprises one or more of pasteurisation, sterilisation, and ultra-high temperature processing (UHT). The enzyme denaturation may comprise pasteurisation, optionally wherein the pasteurisation comprises the application of heat to the mixture comprising mycoprotein cellular materials.

The step of denaturing the enzymes may be carried out after mechanically disrupting the cell walls of the mycoprotein.

The process may comprise the further step of separating the protein from the mixture comprising mycoprotein cellular materials. The step of separating the protein from the mixture comprising mycoprotein cellular materials may be carried out after the enzyme denaturation step. The step of separating the protein from the mixture comprising mycoprotein cellular materials may be carried out in parallel with the enzyme denaturation step.

The alternative features and different embodiments as described apply to each and every aspect and each and every embodiment thereof mutatis mutandis.

### BRIEF DESCRIPTION OF THE DRAWINGS

Embodiments of the invention will now be described, by way of example, with reference to the drawings, in which:

FIG. 1 is a flow diagram which illustrates a process in accordance with one embodiment of the invention;

FIG. 2 is an image of two samples prepared in accordance with the invention and a control sample at 10× magnification;

FIG. 3 is a flow diagram which illustrates a process in accordance with one embodiment of the invention;

FIG. 4 is a flow diagram which illustrates a process in accordance with one embodiment of the invention; and

FIG. 5 is a flow diagram which illustrates a process in accordance with one embodiment of the invention.

### DETAILED DESCRIPTION

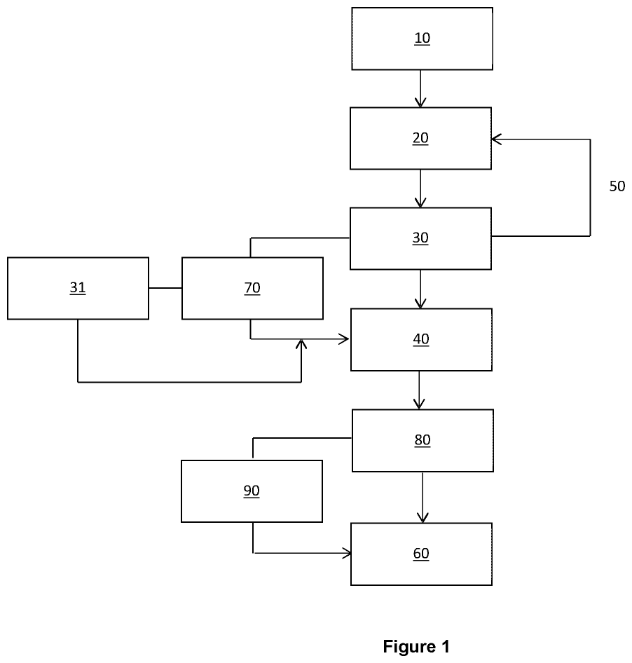

Referring to FIG. 1, there is shown a process for producing mycoprotein and/or the components thereof.

A fermentation media 10 that is rich in glucose is added to the fermentation vessel 20. The fermentation media 10 comprises water, a carbohydrate, a source of nitrogen and nutrients. The carbohydrate is typically glucose. The nutrients are typically selected from salts, vitamins, and trace metals. The salts are typically selected from one or more of the group consisting of potassium sulphate, potassium phosphate, magnesium sulphate, manganese chloride, calcium acetate, calcium chloride, iron sulphate, iron chloride, zinc sulphate, zinc chloride, copper sulphate, copper chloride, cobalt chloride, ammonium chloride, sodium molybdate, ammonium hydroxide and ammonium phosphate. Other components that are optionally added to the fermentation media include, but are not limited to, biotin, choline, and phosphoric acid.

The fermentation media 10 is cooled to 30° C. and inoculated with a mycoprotein-producing microorganism. The temperature used can be from approximately 26° C. to approximately 32° C. The mycoprotein-producing microorganism is a filamentous fungi, optionally from the Fusarium species, and is typically Fusarium venenatum.

Aerobic conditions are maintained by aerating and agitating the media.

The product of the aerobic fermentation is a mixture comprising mycoprotein and at least partially spent fermentation media. To the extent that the fermentation media 10 is not completely spent, the so-formed partially spent fermentation media 50 comprises nutrients and glucose from the fermentation media 10.

The mixture comprising mycoprotein and partially spent fermentation media undergoes a separation step 30, where the mixture is separated into a mycoprotein phase and a partially spent fermentation media phase. The separation step 30 may be performed by any solid-liquid separation means and/or apparatus known in the art. For example, centrifugation (e.g., decanter and/or disc stack centrifugation), filtration (e.g., cross flow filtration), or the like. As illustrated in FIG. 1, at least a portion of the separated partially spent fermentation media 50 is optionally reintroduced into the fermentation vessel 20 or alternatively is disposed of as waste effluent.

The concentration of mycoprotein in the mycoprotein phase may be up to 20 times the concentration of mycoprotein in the mixture comprising mycoprotein and partially spent fermentation prior to separation step 30. Therefore, after separation step 30, the mycoprotein phase is viscous and may have a mycoprotein content of between approximately 1.5% w/w and approximately 30% w/w mycoprotein, and typically is between 10% w/w and approximately 20% w/w mycoprotein. The mycoprotein phase can undergo an optional wash step 70. The wash step 70 typically comprises adding the mycoprotein phase to a buffer (wash) tank and adding water.

If need be, to obtain the desired concentration, after the wash step 70 there is carried out a water removal step 31, similar to the separation that described above (i.e., separation step 30). The optional wash step 70 effectively replaces any residual partially spent fermentation media 50 in the mycoprotein phase with water, thereby mitigating the presence of residual partially spent fermentation media 50 in the mycoprotein phase. After the wash step 70 (and, if carried out, the optional water removal step 31) the concentration of mycoprotein in the mycoprotein phase may be between approximately 1.5% w/w and approximately 30% w/w mycoprotein, and typically is between approximately 10% w/w and approximately 20% w/w.

Irrespective of whether optional wash step 70 is carried out, and as noted above, after separation the mycoprotein phase is a viscous non-Newtonian, non-gravity settling fluid and may have a mycoprotein content of between 1.5% w/w and 30% w/w mycoprotein, and typically is between 10% w/w and 20% w/w. It can be difficult to process such a viscous mixture using cell disruption techniques. Therefore, a viscosity reduction step 40 is performed. The viscosity reduction step 40 is carried out using high shear mixing. This causes the mycoprotein phase to become more Newtonian in nature and also reduces the mycoprotein hyphal length. The high shear mixing applies a mechanical shearing force to the mycoprotein phase, therefore breaking up the hyphal structure and reducing the hyphal length, which in turn causes a reduction in viscosity. Typically, the hyphal length is reduced to less than approximately 50 μm. Techniques that can be used to reduce the viscosity and/or reduce hyphal length of the mycoprotein include high shear mixing and/or blending. An alternative technique that can be used to reduce the viscosity and/or reduce hyphal length of the mycoprotein is the application of thinning agents to the mycoprotein phase.

After the separation step 30 or after optional viscosity reduction step 40 if it is carried out, the mycoprotein phase undergoes a mechanical cell disruption step 80, in which the cells walls of the mycoprotein cells are mechanically disrupted thereby releasing the contents of the mycoprotein cells. Mechanically disrupting the cell walls of the mycoprotein can use mechanical force, such as a shearing force. Examples of techniques that are used for mechanical cell disruption include high pressure homogenisation, microfluidisation, French pressure cell press, sonication, ultrasonication, bead mills, Hughes press and X-press. It is noted that high pressure homogenisation and microfluidisation are useful in the present process, and that high pressure homogenisation is useful on a commercial scale.

On undergoing the mechanical cell disruption step 80 and releasing the cell contents of the mycoprotein there is formed a mixture comprising mycoprotein cellular materials, the mycoprotein cellular materials comprising one or more of: protein and RNA, chitin, glucans, chitosans, nucleic acids, sterols, amino acids, lipids, glycerides, vitamins, enzymes, organic acids, peptides, protein fragments, storage carbohydrates and minerals. The mechanical disruption of the cell walls also inactivates the organism, the fungal cell no longer being viable.

The total cellular RNA originally present in mycoprotein is typically approximately 10% w/w on a dry weight basis. Releasing the RNA from the mycoprotein cells causes denaturing of the RNA so that the amount of RNA is reduced to less than approximately 2% w/w on a dry weight basis. In some embodiments, the amount of RNA is reduced to less than approximately 1% w/w on a dry weight basis. It should be understood that more than approximately 8% w/w (or more than approximately 9% w/w) RNA is denatured, having been released from the mycoprotein cells, meaning that the remaining (i.e., less than approximately 2% (or 1%)) w/w RNA is RNA that is viable or not denatured. On denaturing of the RNA there is provided mycoprotein and/or the components thereof 60 that are usable as or in foodstuffs.

After mechanical cell disruption 80 there is an optional mycoprotein isolate separation step 90, wherein one or more of the components of the mixture comprising mycoprotein cellular materials is isolated. For example, the protein may be separated from the mixture comprising mycoprotein cellular materials. The protein can be further treated by proteolysis to provide its constituent amino acids. This optional step provides mycoprotein components 60 that are usable as or in foodstuffs.

Following mechanical cell disruption 80 and/or the optional mycoprotein isolate separation step 90, there is provided mycoprotein and/or the components thereof 60 such as, for example, mycoprotein cellular materials and the constituent parts of those cellular materials. The mycoprotein and/or the components thereof 60 is a non-fibrous composition and has an RNA content of less than 2% w/w, typically less than 1% w/w on a dry weight basis.

Any waste product from the optional mycoprotein isolate separation step 90 can be separated and disposed or discharged from the process as waste effluent.

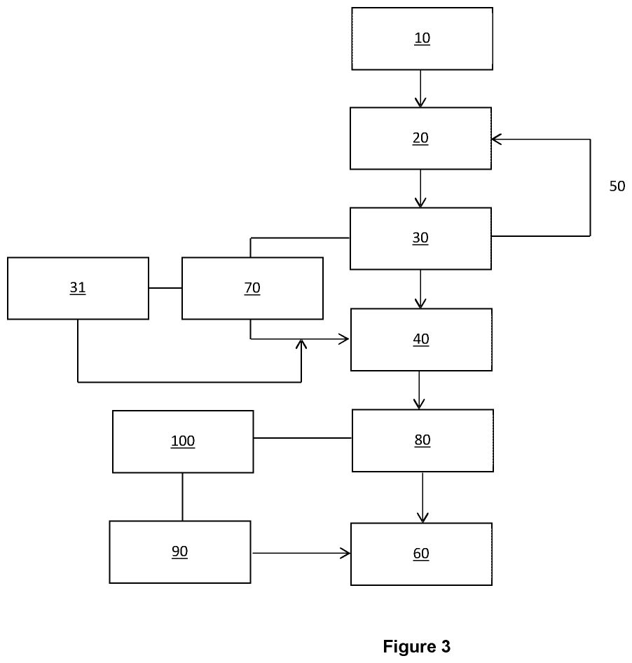

Referring to FIG. 3, there is shown one embodiment of the invention, wherein after mechanical cell disruption 80 there is an optional enzyme denaturation step 100. In the enzyme denaturation step 100, the enzymes present in the mixture comprising mycoprotein cellular materials are denatured, such that they are no longer functional. For example, tyrosinase may be denatured, such that it can no longer break down tyrosine to form melanin. The enzyme denaturation may be performed by enzyme denaturation techniques known in the art. For example, the enzyme denaturation step 100 may comprise pasteurisation. That is, the mixture comprising mycoprotein cellular materials can be pasteurised. The pasteurisation comprises the application of heat to the mixture comprising mycoprotein cellular materials. Typically, for pasteurisation, the mixture is heated at a temperature of approximately 70° C. for approximately 15 minutes. However, the mixture can be heated at a temperature of between approximately 70° C. and approximately 135° C. for between approximately 30 seconds and approximately 30 minutes. The enzyme denaturation 100 may comprise one or more of pasteurisation, sterilisation, and ultra-high temperature processing (UHT). Typically, for sterilisation, the mixture can be heated at a temperature of approximately 121° C. for approximately 30 minutes. Typically, for UHT, the mixture can be heated at a temperature of approximately 135° C. for approximately 30 seconds.

The optional enzyme denaturation step 100 is before the optional mycoprotein isolate separation step 90, wherein one or more of the components of the mixture comprising mycoprotein cellular materials is isolated.

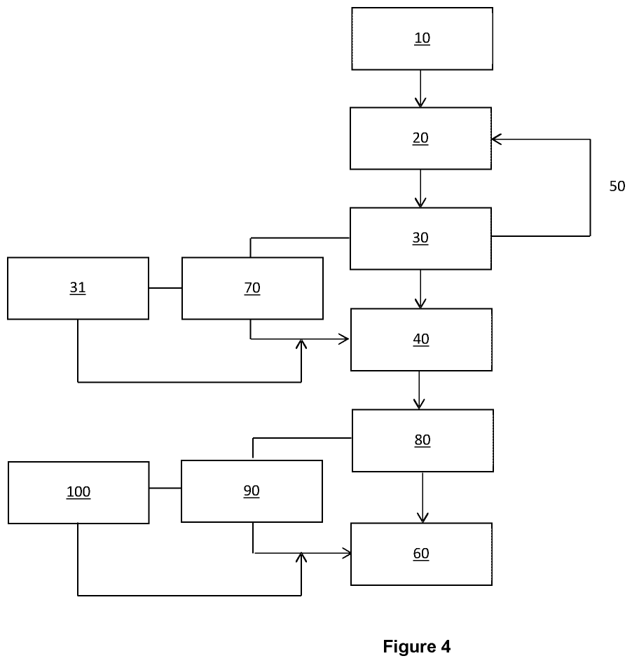

Referring to FIG. 4, there is shown one embodiment of the invention, wherein after mechanical cell disruption 80 there is an optional enzyme denaturation step 100. In this embodiment, the optional enzyme denaturation step 100 is carried out in parallel with the optional mycoprotein isolate separation step 90, wherein one or more of the components of the mixture comprising mycoprotein cellular materials is isolated.

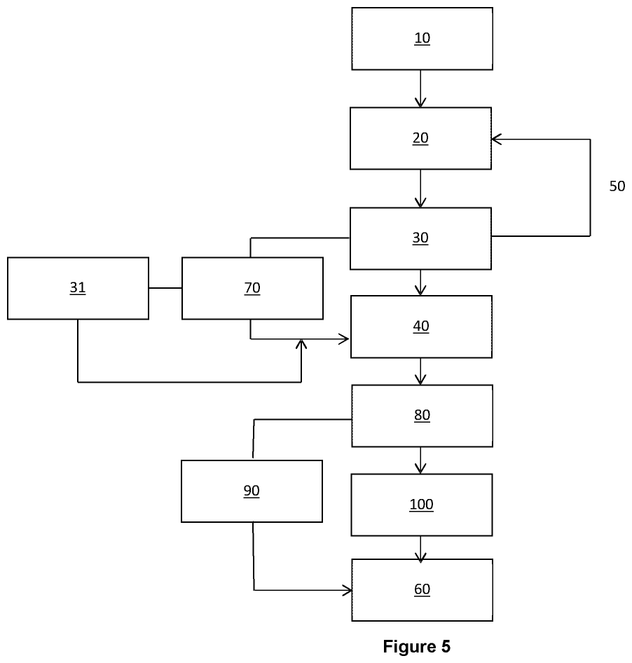

Referring to FIG. 5, there is shown one embodiment of the invention, wherein after mechanical cell disruption 80 there is an optional enzyme denaturation step 100. In this embodiment, the optional enzyme denaturation step 100 is carried out after the mechanical cell disruption step 80, in which the cells walls of the mycoprotein cells are mechanically disrupted thereby releasing the contents of the mycoprotein cells.

The mechanical cell disruption step 80 inactivates the microorganism used for fermentation, such that the fungal cell is no longer viable. This means that only enzymes that have already been transcribed will be present in the mixture comprising mycoprotein cellular materials. As such, expression of new proteases (enzymes that break down protein biomass) will not occur. Therefore, the application of heat in the enzyme denaturation step 100 will not result in the same loss of mycoprotein biomass observed during the heat treatment step that is conventionally used to reduce RNA content (where heat is applied when the cells are still viable and new proteases are expressed).

When the RNA is released from the mycoprotein cell it immediately begins to degrade. This is because outside of the mycoprotein cell, the RNA is inherently unstable. Denaturing of the RNA can also be caused by the action of ribonuclease enzymes that have also been released from the mycoprotein cells or other factors such as the interaction of the RNA with UV light or with other components of the mixture comprising mycoprotein cellular materials.

In the present process, the denaturing of the RNA occurs without the application of heat. For example, the denaturing of the RNA can occur at typical ambient temperatures (e.g., up to 25° C.), but of course will also occur if the ambient temperature happens to be higher (e.g., up to 40° C.). There is no requirement, however, for the application of heat from an external heat source to denature the RNA. As noted above, when the RNA is released from the mycoprotein cell it immediately begins to degrade. However, in some instances, the mixture comprising mycoprotein cellular materials may be allowed to sit for a period of time to ensure that RNA content has reduced to a suitable level. For example, the mixture could be allowed to sit for up to approximately 60 minutes at ambient temperature to allow the RNA to denature, or the mixture could be allowed to sit for up to approximately 15 minutes.

The advantage of denaturing the RNA without the application of heat is that mycoprotein biomass loss is mitigated. Typically, around 30% of the mycoprotein just produced is lost in the heat step that is normally used to reduce RNA content. Furthermore, the heat step is energy intensive. The use of mechanical cell disruption to enable denaturing of RNA avoids the use of heat and mitigates mycoprotein biomass loss caused by heating.

Previously, mechanical cell disruption was not used in the preparation of mycoprotein for use in foodstuffs as those foodstuffs typically require the fibrous structure associated with mycoprotein, and that fibrous structure is lost in the present process. The loss of fibrous structure is due to both high shear mixing (used to reduce viscosity) and high-pressure homogenisation (or other mechanical cell disruption techniques).

The following experiments were performed by way of exemplification.

Experiment 1

A fermentation media 10 is prepared by adding the nutrients outlined in Table 1 to 195 L of deionised water.

TABLE 1
Nutrient Composition of Fermentation Media

| Fermentation Media Component | Concentration (g/L) |
|---|---|
| Potassium sulphate (K₂SO₄) | 2 |
| Magnesium sulphate heptahydrate (MgSO₄ · 7H₂O) | 0.9 |
| Calcium Acetate (Ca(C₂H₃O₂)₂) | 0.2 |
| Phosphoric Acid (85%) | 1.15 (mL/L) |
| Iron (II) sulphate heptahydrate (FeSO₄ · 7H₂O) | 0.005 |
| Zinc sulphate heptahydrate | 0.025 |
| Manganese sulphate tetrahydrate (MnSO₄ · 4H₂O) | 0.02 |
| Copper sulphate heptahydrate (CuSO₄ · 7H₂O) | 0.0025 |
| Biotin (C₁₀H₁₆N₂O₃S) | 0.000025 |
| Choline chloride (C₅H₁₄ClNO) | 0.087 |
| Glucose (C₆H₁₂O₆) | 33 |

Fermenter vessel 20 is sterilised empty (30 minutes at >121° C., 18-20 PSI). The pH probe is installed and calibrated pre-sterilisation and the DO2 probe is membrane checked, electrolyte changed and installed. 100% DO2 calibration is carried out post sterilization after the vessel has been left to stabilize (at 1 VVM airflow and 200 rpm) for at least 6 hours.

Before sterilisation, care is taken to carefully secure all connections in the fermentation vessel 20; for example, all addition ports are secured using o-ring gaskets and tri clamp fittings and respective collar fittings.

After sterilisation and after the fermentation vessel 20 is cooled down to an ambient temperature, feed as per Table 1 is pumped into fermentation vessel 20 under aseptic conditions using a peristatic pump through sterile tubing and filter (0.2 um) (Sartorius Midicap). The feed contains all nutrient components including Biotin and Choline chloride. Thereafter, the pH of the fermentation media 10 is adjusted to pH 6.0 using a suitable base (in this example 35% Ammonium Hydroxide is used, but 28% to 35% Ammonium Hydroxide can be used).

A dissolved oxygen (DO) probe is inserted into the fermentation vessel 20 before sterilisation. The probe is then calibrated after sterilisation. The DO probe is calibrated at a fermentation temperature of 30° C., with an air flow of 200 L/min (1 VVM (volume of air per volume of liquid per minute)) and stirring speed of 200 rpm and the DO probe is set to 100%. The air enters the fermentation vessel 20 through a sterile inlet filter and sparger. Air escapes into the vessel headspace and then is passed to atmosphere through pressure sensor valve.

Fermentation is initiated by adding 5 L of 0.5% w/v inoculum (Fusarium venenatum in deionised water) into the fermentation vessel 20. The volume can be 1 L to 5 L. This gives a final fermentation media 10 volume of 200 L and an inoculum concentration of 0.125% v/v. The initial biomass can be from 5 to 25 gram in 200 L=0.025 to 0.125 g/L. Fermentation is carried out under a controlled aerobic environment at 30° C., with dissolved oxygen level (DO-30%) maintained using variable agitation (200 to 450 rpm) and aeration (1 to 1.8 WM). During fermentation, ammonium hydroxide (35%) is used for both pH control and as a source of nitrogen.

The fermentation is continued until a biomass (mycoprotein) concentration of approximately 14-17 g/L dry weight is achieved. The fermentation is then maintained by adding additional fresh fermentation media (outlined in Table 1) to the fermentation vessel 20 at a rate equal to the growth rate of the microorganism (approximately 0.17-0.2 h⁻¹).

The resulting mixture comprising mycoprotein 60 and partially spent fermentation media 50 is removed from the fermentation vessel 20 for separation 30.

In this experiment, the mycoprotein 60 and partially spent media 50 are separated using a decanter centrifuge (Lemitec, MD80). However, it should be appreciated that other separation techniques may be used. For example, cross-flow filtration, or disc stack centrifuge.

During this step, the liquid phase is continuously collected from the centrifuge whilst the solids are ejected into the decanter chute and captured in a sterile bag. The solids comprise mycoprotein 60 and partially spent fermentation media 50.

The decanter bowl speed and differential speed settings were altered across five samples (see Table 2) with the aim of dewatering the mixture to a solids content of 0% dry solids.

TABLE 2
Decanter Centrifuge Separation (Dewatering)
of Sample 1 to 5 from Experiment 1.

| | Bowl speed | Differential | Thicks | |
|---|---|---|---|---|
| Sample | (rpm) | speed (rpm) | % solids | Amount (kg) |
| 1 | 3000 | 200 | 15 | 0.1 |
| 2 | 3000 | 50 | 13.1 | 0.2 |
| 3 | 4000 | 150 | 12.5 | 0.22 |
| 4 | 4000 | 200 | 12.6 | 0.57 |
| 5 | 3000 | 125 | 10 | 0.41 |

After separation 30, samples 4 and 5 were subject to a viscosity reduction step 40, before cell disruption 80. The volume of samples 1, 2 and 3 was too low to be processed and thus these samples were discarded.

The viscosity reduction step 40 was carried out on samples 4 and 5 by taking all material available and blending with a domestic kitchen blender (600 W) for 60 sec. A Silverson™ high shear blender may also be used.

Cell disruption 80 was then carried out on samples 4 and 5 as follows. High pressure cell disruption was applied to the samples using a microfluidizer as provided by Analytik™. The conditions used were as follows. 100 g of each sample was added to Microfluidizer M-110P™ fitted with chambers H30Z and G10Z. These chambers are a fixed geometry to deliver high shear and thus mechanical damage to the cells. The minimum chamber internal diameter was 50 μm. The shear rate used was 7000 s⁻¹ and the operating pressure was between 20,000 and 30,000 psi (approximately 137 MPa to 207 MPa). The process was carried out at a temperature of approximately 20° C. and at a flow rate between 50-100 mL/min.

The above microfluidizer is an example of high-pressure homogenisation or high-pressure cell disruption, whereby cells are forced through a chamber, which comprises a microchannel, at high pressure. The resulting pressure drop causes the cells to experience mechanical shear thereby causing the cell wall to disrupt at the low pressure outlet. The material passing through the chamber receives consistent, high-shear rates and internal impact forces.

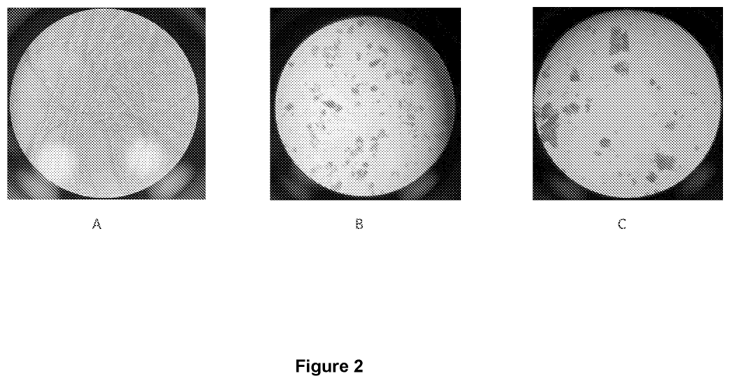

Samples 4 and 5 were passed through the microfluidizer five and three times, respectively. For sample 4, a 50 mL microfluidized sample was collected after the first, third and fifth pass. For sample 5, a 50 mL microfluidized sample was collected after each pass. However, after one pass through the microfluidizer, complete cell disintegration was achieved in the samples (see FIG. 2). Consecutive passes through the microfluidizer showed no further impact on the morphology of the samples.

10 mL of the microfluidized samples were centrifuged at 9,000 rpm for 5 minutes. The clear supernatant was collected in a sterile tube and its volume was measured. Harvested pellets were diluted appropriately using sterile water for slide preparation to observe cell disintegration. A 1 to 20 μL aliquot of dilute sample was placed on a clean microscope slide and smeared with a cover slip to form a thin covering. This was allowed to air dry, flooded with 100% methanol to wash and air dried again. 10 to 15 μL of 40% glycerol was then added and a cover slip placed fully over the smear. The prepared slides were then examined at between 10× to 40× magnification.

After cell disruption, the samples were very smooth and free flowing, unlike the control samples. The mixture comprising mycoprotein and/or mycoprotein cellular materials was analysed using light microscopy and the images produced are shown in FIG. 2. From FIG. 2 it can be seen that the control sample (A) retains a fibrous or filamentous structure, whereas in samples 4 (B) and 5 (C) (after one pass through the cell disruption step (microfluidizer)) complete cell disintegration has been achieved.

Protein Estimation

The protein content in the whole cell lysate (i.e., the samples after cell disruption) was measured using BCA (Bicinchoninic Acid Assay) kit sourced from Fisher™. The results are summarised in Table 3. Protein is present in the fungal cell as an insoluble and a soluble fraction. Calculation in the theoretical column assumes that all the protein in the fungal cell (55.5 gram per 100 gram of dry cell weight) is available as a soluble fraction. Similarly, the experimental column in the same table represents both soluble and insoluble fractions of the protein.

TABLE 3
Protein Content After Cell Disruption
Theoretical Calculations

| | | | | | Theoretical |
|---|---|---|---|---|---|
| | | Sample | | Volume of | protein |
| | | treatment | Amount of | the sample | concentration |
| | Processing | for protein | solids (g/L or | used in | in the sample |
| Sample | method | measurement | kg in DCW) | analysis (L) | (g/L syrup) |
| NA | French Press | None | 20 | 0.01 | 11.1 |
| | (1 Pass) | | | | |
| NA | French Press | | 50 | 0 | 27.8 |
| | (2 Passes) | | | | |
| NA | French Press | | 75 | 0 | 41.6 |
| | (3 Passes) | | | | |
| NA | French Press | | 100 | 0 | 55.5 |
| | (4 Passes) | | | | |
| 4 | Analytik ™ | Only pellet | 126 | 0.01 | 69.9 |
| | Microfluidizer | treated with | | | |
| | (1 Pass) | Yatalase | | | |
| 5 | Analytik ™ | | 100 | 0.01 | 55.5 |
| | Microfluidizer | | | | |
| | (1 Pass) | | | | |
| 4 | Analytik ™ | Whole cell lysate | 126 | 0.005 | 69.93 |
| | Microfluidizer | treated with | | | |
| | (1 Pass) | Yatalase | | | |
| 5 | Analytik ™ | | 100 | 0.005 | 55.5 |
| | Microfluidizer | | | | |
| | (1 Pass) | | | | |

Experimental Value Based on Protein Assay

| | | | | Protein | | Protein |
|---|---|---|---|---|---|---|
| | | Sample | | (g) in the | | recovery |
| | | treatment | | processed | Protein | (experimental |
| | Processing | for protein | Protein | sample | (g)/L/kg | value/theoretical |
| Sample | method | measurement | (g/L) | (10 mL) | of syrup | value) |
| NA | French Press | None | 1.93 | 0.014 | 1.4 | 13% |
| | (1 Pass) | | | | | |
| NA | French Press | | 3.24 | 0.023 | 2.3 | 8% |
| | (2 Passes) | | | | | |
| NA | French Press | | 3.65 | 0.036 | 2.6 | 6% |
| | (3 Passes) | | | | | |
| NA | French Press | | Sample not processed due to high viscosity | | | |
| | (4 Passes) | | | | | |
| 4 | Analytik ™ | Only pellet | 7.21 | 0.032 | 10.8 | 16% |
| | Microfluidizer | treated with | | | | |
| | (1 Pass) | Yatalase | | | | |
| 5 | Analytik ™ | | 6.55 | 0.029 | 16.0 | 29% |
| | Microfluidizer | | | | | |
| | (1 Pass) | | | | | |
| 4 | Analytik ™ | Whole cell | 9.72 | 0.053 | 10.69 | 15% |
| | Microfluidizer | lysate treated | | | | |
| | (1 Pass) | with Yatalase | | | | |
| 5 | Analytik ™ | | 9.13 | 0.050 | 10.05 | 18% |
| | Microfluidizer | | | | | |
| | (1 Pass) | | | | | |

As there was no observed difference in terms of cell disintegration and/or protein concentration in samples that were microfluidized more than once, the data for sample 4 and for sample 5 was pooled and presented as a mean value of three independent experiments.

Based on the results in Table 3 and observations made when carrying out mechanical cell disruption, it seems that mycoprotein samples with a solid concentration above 5% dry cell weight need to undergo viscosity reduction by high shear mixing before they undergo cell disruption by high pressure homogenisation. Doing so reduces the viscosity of the mycoprotein phase (which is in the form of a paste) and makes it more free flowing. Viscosity reduction also reduces the chances of microfluidics channel blockage. It also appears that a mycoprotein phase (in the form of a paste) with a solid concentration above 15% dry cell weight may be difficult to process through a microfluidizer, even after initial viscosity reduction.

Again, based on the results in Table 3 and observations made when carrying out mechanical cell disruption, it is thought that microfluidization inflicts greater cell damage than the French press; this is reflected in the amount of soluble protein present in the supernatant. For example, a sample generated using microfluidics showed higher protein level (4×) and protein recovery compared to sample generated using a French press. Furthermore, complete cell disintegration was achieved after passing samples just once through a microfluidizer—this was not the case when using the French press. Note that the samples used in the French press were prepared in a similar way to samples 1 to 5.

RNA Estimation

The RNA content of the whole cell lysate (i.e., the samples after cell disruption) was measured using a modified Bial's method and results are summarised in Table 4. For the method 2 grams of fungal biomass is washed with acetone (20 ml) and then filtered. Thereafter, 100 mg of the washed biomass is suspended in 4 mL of enzyme solution (10 g/L Yatalase (Takara) in Acetate Buffer). The solution is then incubated overnight (>12 hours) in a shaker at 37° C. and 100 rpm. After incubation samples are centrifuged for 5 min at 10,000 rpm and 10-500 μL supernatant decanted into sterile RNase free Eppendorf tubes. 500 μL of freshly prepared Bial's reagent (15 parts 0.05% FeCl₃·6H₂O in concentrated HCl mixed with 1 part 422 mM orcinol monohydrate in 95% ethanol) is added to each tube, mixed and then incubated in a boiling water bath for 20 min. The samples are then cooled and centrifuged for 5 min at 10,000 rpm. RNA content is then calculated by measuring absorbance at 660 nm using a spectrophotometer.

The total RNA in the fungal cell is present in the cytoplasmic compartment, and after cell disruption (cell breakdown) is present in the liquid fraction. Calculation in the theoretical column in Table 4 assumes that all of the RNA in the fungal cell (10 gram per 100 gram of dry cell weight) is available in the liquid as a soluble fraction. Similarly, the experimental column in Table 4 represents only the soluble fraction of the mycoprotein.

TABLE 4
RNA Present After Cell Disruption

| | | | | | RNA (g) in | |
|---|---|---|---|---|---|---|
| | | Sample | Amount | Theoretical RNA | solid at the | |
| | | treatment for | of solids | concentration in | concentration | |
| | Processing | protein | (g/L or kg | the sample | used or per | % RNA |
| Sample | method | measurement | in DCW) | DCW (g/L or kg) | L of sample | reduction |
| NA | French Press | None | 20 | 2 | Not analysed due | |
| | (1 Pass) | | | | to sample limitation | |
| NA | French Press | | 50 | 5 | | |
| | (2 Passes) | | | | | |
| NA | French Press | | 75 | 7.5 | | |
| | (3 Passes) | | | | | |
| NA | French Press | | 100 | 10 | | |
| | (4 Passes) | | | | | |
| 4 | Analytik ™ | Only pellet | 126 | 12.6 | 0.52 | 96% |
| | Microfluidizer | treated with | | | | |
| | (1 Pass) | Yatalase | | | | |
| 5 | Analytik ™ | | 100 | 10 | 0.42 | 96% |
| | Microfluidizer | | | | | |
| | (1 Pass) | | | | | |
| 4 | Analytik ™ | Whole cell | 126 | 12.6 | 0.01 | 100% |
| | Microfluidizer | lysate treated | | | | |
| | (1 Pass) | with Yatalase | | | | |
| 5 | Analytik ™ | | 100 | 10 | 0.11 | 99% |
| | Microfluidizer | | | | | |
| | (1 Pass) | | | | | |

Based on the results in Table 4, the total RNA in protein syrup (1 L) with solid concentration 126 and 100 gram (dry cell weight) corresponds to 1.72 and 3.7 grams, respectively. Therefore, it is observed that without any heat treatment, cell disruption releases cytoplasmic RNA into the liquid phase, which is denatured and/or degrades into smaller components by the action of ribonuclease enzymes that have also been released from the mycoprotein cells or other factors such as the interaction of the RNA with UV light or with other components of the mixture comprising mycoprotein cellular materials.

The products prepared by the present process typically comprise the nutritional components listed in Table 5. Note that the quantities given are averages and are not intended to be limiting.

TABLE 5
Typical Nutritional Components of the Mycoprotein (25% solids)

| Analyte (per 100 g wet basis) | Average | |
|---|---|---|
| Total Carbohydrates | 4.7 | g |
| Total Fat | 1.7 | g |
| Sodium | 1 | mg |
| Calcium | 24 | mg |
| Copper | 0.4 | mg |
| Iron | 0.5 | mg |
| Magnesium | 24 | mg |
| Manganese | 1.5 | mg |
| Phosphorus | 185 | mg |
| Potassium | 101 | mg |
| Zinc | 11 | mg |
| Thiamine (B1) | 0.02 | g |
| Riboflavin (B2) | 0.16 | g |
| Niacin (B3) | 0.38 | g |
| Pantothenic acid (B5) | 0.18 | g |
| Biotin | 0.05 | g |
| Folate | 0.03 | g |

Where proteolysis has taken place, the products prepared by the present process typically comprise the amino acids listed in Table 6. Note that the quantities given are averages and are not intended to be limiting.

TABLE 6
Typical Amino Acids from the Proteolysis
of Protein Obtained from Mycoprotein

| Amino acid (g/100 g protein) | Average |
|---|---|
| Alanine | 6.5 |
| Arginine | 6.9 |
| Aspartic acid | 10.4 |
| Glutamic acid | 11.9 |
| Glycine | 4.7 |
| Histidine | 2.5 |
| Isoleucine | 4.9 |
| Leucine | 7.7 |
| Lysine | 7.7 |
| Phenylalanine | 4.7 |
| Proline | 5.0 |
| Serine | 4.9 |
| Threonine | 5.2 |
| Tyrosine | 3.1 |
| Valine | 5.9 |
| Cystein & Cystine | 0.8 |
| Methionine | 2.0 |
| Tryptophan | 1.4 |

Experiment 2

A fermentation media 10 (195 L) is prepared as described in Experiment 1.

Fermenter vessel 20 is sterilised empty (30 minutes at >121° C., 18-20 PSI). The pH probe is installed and calibrated pre-sterilisation and the DO2 probe is membrane checked, electrolyte changed and installed. 100% DO2 calibration is carried out post sterilization after the vessel has been left to stabilize (at 1 VVM airflow and 200 rpm) for at least 6 hours.

Before sterilisation, care is taken to carefully secure all connections in the fermentation vessel 20; for example, all addition ports are secured using o-ring gaskets and tri clamp fittings and respective collar fittings.

After sterilisation and after the fermentation vessel 20 is cooled down to an ambient temperature, feed as per Table 1 is pumped into fermentation vessel 20 under aseptic conditions using a peristatic pump through sterile tubing and filter (0.2 um). The feed contains all nutrient components including Biotin and Choline chloride. Thereafter, the pH of the fermentation media 10 is adjusted to pH 5.9 using a suitable base (in this example 35% Ammonium Hydroxide is used, but 28% to 35% Ammonium Hydroxide can be used).

A dissolved oxygen (DO) probe is inserted into the fermentation vessel 20 before sterilisation. The probe is then calibrated after sterilisation. The DO probe is calibrated at a fermentation temperature of 30° C., with an air flow of 200 L/min (1 VVM (volume of air per volume of liquid per minute)) and stirring speed of 200 rpm and the DO probe is set to 100%. The air enters the fermentation vessel 20 through a sterile inlet filter and sparger. Air escapes into the vessel headspace and then is passed to atmosphere through pressure sensor valve.

Fermentation is initiated by adding 4.5 L of 0.5% w/v inoculum (Fusarium venenatum in deionised water) into the fermentation vessel 20. The volume can be 1 L to 5 L. This gives a final fermentation media 10 volume of 200 L and an inoculum concentration of 0.113% v/v. The initial biomass can be from 5 to 25 gram in 200 L=0.025 to 0.125 g/L. Fermentation is carried out under a controlled aerobic environment at 30° C., with dissolved oxygen level (DO-30%) maintained using variable agitation (200 to 450 rpm), pressure (8 to 18 psi) and aeration (200 to 360 L/min). During fermentation, ammonium hydroxide (35%) is used for both pH control and as a source of nitrogen.

The fermentation is continued until a biomass (mycoprotein) concentration of approximately 12-16 g/L dry weight is achieved. The fermentation is then maintained by adding additional fresh fermentation media (outlined in Table 1) to the fermentation vessel 20 at a rate equal to the growth rate of the microorganism (approximately 0.16-0.2 h⁻¹).

The resulting mixture comprising mycoprotein 60 and partially spent fermentation media 50 is removed from the fermentation vessel 20 for separation 30.

In this experiment, the mycoprotein 60 and partially spent media 50 are separated using a decanter centrifuge (Lemitec, MD80). However, it should be appreciated that other separation techniques may be used. For example, cross-flow filtration, or disc stack centrifuge.

During this step, the liquid phase is continuously collected from the centrifuge whilst the solids are ejected into the decanter chute and captured in a sterile bag. The solids comprise mycoprotein 60 and partially spent fermentation media 50.

The decanter bowl speed and differential speed settings were altered across seven samples (see Table 7) with the aim of dewatering the mixture to a solids content of ≤10% dry solids.

TABLE 7
Decanter Centrifuge Separation (Dewatering)
of Sample 1 to 7 from Experiment 2.

| | Bowl speed | Differential | Thicks | |
|---|---|---|---|---|
| Sample | (rpm) | speed (rpm) | % solids | Amount (kg) |
| 1 | 1000 | 100 | 0.9 | 5.1 |
| 2 | 2000 | 50 | 2.9 | 5 |
| 3 | 3000 | 25 | 8.9 | 5.7 |
| 4 | 3000 | 10 | 9.0 | 5.7 |
| 5 | 4000 | 100 | 7.7 | 3.05 |
| 6 | 3000 | 50 | 6.8 | 5.8 |
| 7 | 3000 | 100 | 6.2 | 3.19 |

After separation 30, samples 3 and 7 were processed as outlined below. Samples 1, 2, 4, 5 and 6 were not processed.

Samples 3 and 7 were packed in food grade bags, sealed, and stored at −20° C. until required.

Sample bags collected contained live biomass and continued respiration (until the bags were frozen). This inflated the bags. Therefore, before processing, the bags were punctured using a sterile needle and allowed to defrost overnight in a cabinet to avoid contamination.

The viscosity reduction step 40 was carried out on sample 3 by taking all material available and blending with a domestic kitchen blender (600 W) for 2 to 3 minutes to achieve homogeneity. A Silverson™ high shear blender may also be used. However, this incorporated air into the sample making it difficult to process using Microfluidics. For this reason, sample 7 did not undergo a viscosity reduction step 40, but was mixed gently by swirling the bags or bottles to avoid incorporating air into the sample. Sample 3 was placed in a water bath with the temperature set to 30° C. before further processing.

Cell disruption 80 was then carried out on samples 3 and 7 as follows. High pressure cell disruption was applied to the samples using a microfluidizer as provided by Analytik™. The conditions used were as follows. 100 g of each sample was added to Microfluidizer M-110P™ fitted with chambers H30Z and G10Z. These chambers are a fixed geometry to deliver high shear and thus mechanical damage to the cells. The minimum chamber internal diameter was 50 μm. The shear rate used was 7000 s⁻¹ and the operating pressure was between 10,000 and 30,000 psi (approximately 68 MPa to 207 MPa). The sample processing temperature was maintained between 20 to 30° C. by flushing the cooling block regularly with cold tap water. Samples were pumped at a flow rate of 100 m L/m in.

The above microfluidizer is an example of high-pressure homogenisation or high-pressure cell disruption, whereby cells are forced through a chamber, which comprises a microchannel, at high pressure. The resulting pressure drop causes the cells to experience mechanical shear thereby causing the cell wall to disrupt at the low pressure outlet. The material passing through the chamber receives consistent, high-shear rates and internal impact forces.

Sample 3 was exposed to a pressure of 30,000 psi and passed consecutively three times through the microfluidizer. Sample 7 was subjected to a low shear test at a pressure of 10,000 psi and 20,000 psi with three passes at both pressures.

Protein Estimation

The protein content in the whole cell lysate (i.e., the samples after cell disruption) was measured using BCA (Bicinchoninic Acid Assay) kit sourced from Fisher™. The results are summarised in Table 8. Protein is present in the fungal cell as an insoluble and a soluble fraction. Pass 3 of samples 3 and 7 were centrifuged and separated into pellet and supernatant (SN) fractions to determine the concentration of soluble versus insoluble protein as a determination of cell breakage efficiency when compared against the total protein (homogenate).

TABLE 8
Protein Content After Cell Disruption

| | | | | | | Protein | |
|---|---|---|---|---|---|---|---|
| | Pressure | Pass | | | | Concentration | % of total |
| Sample | (psi) | No. | Fraction | Abs. | Dilution | (g/L) | protein |
| 7 | 20,000 | 1 | Homogenate | 0.354 | 100 | 14.3 | |
| | | 2 | Homogenate | 0.313 | 100 | 11.2 | |
| | | 3 | Homogenate | 0.342 | 100 | 13.4 | 100 |
| | | | pellet | 1.161 | 10 | 7.5 | 55.8 |
| | | | (insoluble) | | | | |
| | | | SN (soluble) | 0.919 | 10 | 5.6 | 42.3 |
| | 10,000 | 1 | Homogenate | 0.449 | 100 | 21.5 | |
| | | 2 | Homogenate | 0.35 | 100 | 14.0 | |
| | | 3 | Homogenate | 0.357 | 100 | 14.5 | |
| 3 | 30,000 | 3 | Homogenate | 0.427 | 100 | 19.8 | 100 |
| | | | pellet | 1.457 | 10 | 9.7 | 49.0 |
| | | | (insoluble) | | | | |
| | | | SN (soluble) | 1.508 | 10 | 10.1 | 51.0 |

The data shows good consistency of protein concentration across the runs. On sample 7, a slight decrease in protein concentration is observed with the number of passes at 10,000 psi, but at 20,000 psi this is less significant. On this analysis any difference in operating pressure is limited. The material that has been separated into pellet and supernatant (SN) for samples 7 and 3 is comparable in protein content.

Additional protein analysis was carried out on sample 3 using the Kjeldahl method. The Kjeldahl method involves a three-step approach to the quantification of protein: digestion, distillation, and titration. Digestion of organic material is achieved using concentrated H₂SO₄, heat, K₂SO₄ (to raise the boiling point), and a catalyst (e.g., selenium) to speed up the reaction. This process converts any nitrogen in the sample to ammonium sulfate. The digestate is neutralized by the addition of NaOH, which converts the ammonium sulfate to ammonia, which is distilled off and collected in a receiving flask of excess boric acid, forming ammonium borate. The residual boric acid is then titrated with a standard acid with the use of a suitable end-point indicator to estimate the total nitrogen content of the sample. Following determination of the total nitrogen, the use of a specific conversion factor is needed to convert the measured nitrogen content to the crude protein content. A nitrogen-to-protein conversion factor of 6.25 was used.

The results were as follows:

Nitrogen: 0.52 g/100 g

Protein (Nitrogen*6.25): 3.3 g/100 g

The results confirm availability of protein through this method.

RNA Estimation

The whole cell lysate (i.e., sample after cell disruption) of sample 3 was analysed to determine the RNA content using the following method. A test portion of sample 3 is weighed into a polythene centrifuge tube. RNA present is hydrolysed by perchloric acid and heat, liberating its constituent purine and pyrimadine bases. Any interfering materials are removed by centrifuging. The concentration of liberated bases are measured using a spectrophotometer at 260 nm and compared to a range of RNA standards. The RNA present is determined from the calibration curve.

The results are reported to the nearest 0.1 g/100 g and are summarised in Table 9.

TABLE 9
RNA Analysis After Cell Disruption

| Analysis | Result (g/100 g) |
|---|---|
| Moisture | 93.8 |
| Dry Matter | 6.2 |
| RNA in Dry Matter | 7.2 |
| RNA | 0.45 |

Based on the results in Table 9, the total RNA in protein syrup (1 L) with solid concentration 62 gram (dry cell weight) corresponds to 4.5 grams. Therefore, it is observed that without any heat treatment, cell disruption releases cytoplasmic RNA into the liquid phase, which is denatured and/or degrades into smaller components by the action of ribonuclease enzymes that have also been released from the mycoprotein cells or other factors such as the interaction of the RNA with UV light or with other components of the mixture comprising mycoprotein cellular materials.

The process described herein enables RNA reduction without the application of heat. This mitigates the loss of mycoprotein biomass that occurs when using a heat step to reduce RNA content, which is typically around 30% of the mycoprotein just produced. Furthermore, the heat step is energy intensive. The use of mechanical cell disruption to enable denaturing of RNA avoids the use of heat and mitigates mycoprotein biomass loss caused by heating. In addition, the use of the process as described herein provides a non-fibrous mycoprotein suitable for use in a variety of foodstuffs, including those other than meat substitutes.

The improved process as described herein provides an efficient, cost effective process for obtaining mycoprotein, non-fibrous mycoprotein and/or other products comprising the components of mycoprotein such as, for example, protein and the amino acids form the proteolysis thereof. Overall, the process described herein results in a more efficient, cost effective, versatile, and environmentally friendly process for producing mycoprotein and the components, isolates and products derived therefrom.

While this invention has been described with reference to the sample embodiments thereof, it will be appreciated by those of ordinary skill in the art that modifications can be made to the structure and elements of the invention without departing from the spirit and scope of the invention as a whole.

1. A process for producing at least one of mycoprotein and the components thereof, the process comprising:
(i) providing a fermentation media suitable for producing mycoprotein;
(ii) fermenting the fermentation media to obtain a mixture comprising mycoprotein;
(iii) separating the mycoprotein from the mixture to obtain a mycoprotein phase; and
(iv) mechanically disrupting the cell walls of the mycoprotein in the mycoprotein phase thereby releasing at least some of the mycoprotein cell contents.

2. The process of claim 1, wherein the process comprises:
(i) providing a fermentation media suitable for producing mycoprotein;
(ii) introducing the fermentation media to a fermentation vessel;
(iii) fermenting the fermentation media to obtain a mixture comprising mycoprotein and at least partially spent fermentation media;
(iv) separating the mixture comprising mycoprotein and at least partially spent fermentation media into a mycoprotein phase and an at least partially spent fermentation media phase; and
(v) mechanically disrupting the cell walls of the mycoprotein in the mycoprotein phase thereby releasing at least some of the mycoprotein cell contents.

3. (canceled)

4. The process of claim 1, wherein mechanically disrupting the cell walls comprises using one or more of: high pressure homogenisation, microfluidisation, a French pressure cell press, sonication, ultrasonication, bead mills, a Hughes press and an X-press.

5. The process of claim 1, wherein the mycoprotein in the mycoprotein phase comprises cell walls, cell contents, and at least one hypha having a hyphal length.

6. The process of claim 1, wherein the process comprises the further step of reducing the viscosity of the mycoprotein phase.

7. The process of claim 6, wherein the mechanical disruption of the cell walls is carried out after the reduction in viscosity of the mycoprotein phase.

8. (canceled)

9. The process of claim 6, wherein the step of reducing the viscosity of the mycoprotein phase comprises reducing the hyphal length of the mycoprotein in the mycoprotein phase.

10. The process of claim 1, wherein mechanically disrupting the cell walls of the mycoprotein in the mycoprotein phase is carried out without the application of heat.

11. The process of claim 1, wherein on releasing the cell contents of the mycoprotein in the mycoprotein phase, there is formed a mixture comprising mycoprotein cellular materials, the mycoprotein cellular materials comprising one or more of: protein and RNA.

12. The process of claim 11, wherein the mycoprotein cellular materials comprise other cellular materials, the other cellular materials comprising one or more of: chitin, glucans, chitosans, nucleic acids, sterols, amino acids, lipids, glycerides, vitamins, enzymes, organic acids, peptides, protein fragments, storage carbohydrates and minerals.

13. The process of claim 12, wherein the process comprises the further step of denaturing the enzymes in the mixture comprising mycoprotein cellular materials.

14. The process of claim 11, wherein the process comprises the further step of separating the protein from the mixture comprising mycoprotein cellular materials.

15. The process of claim 11, wherein the process comprises the further step of proteolysis of the protein into constituent amino acids.

16. The process of claim 11, wherein releasing the RNA from the mycoprotein cells causes denaturing of the RNA.

17. The process of claim 16, wherein the denaturing of the RNA occurs without the application of heat.

18. The process of claim 16, wherein the denaturing of the RNA occurs at ambient temperature, where ambient temperature is up to approximately 40° C.

19. (canceled)

20. (canceled)

21. (canceled)

22. The process of claim 1, wherein the process comprises the further step of washing the mycoprotein phase with water so-forming a mixture comprising mycoprotein and water, wherein the wash step is carried out after the separation step and before the mechanical cell disruption step.

23. The process of claim 22, wherein the process comprises the further step of removing water from the so-formed mixture comprising mycoprotein and water.

24. Mycoprotein obtainable, obtained or directly obtained by the process of claim 1.

25. A composition comprising at least one of mycoprotein and the components thereof, wherein the composition is non-fibrous.
